(19) United States
(12) Patent Application Publication
(10) Pub. No.: US 2008/0050316 A1
(43) Pub. Date: Feb. 28, 2008

(54) MOLECULAR IMAGING OF EPITHELIAL CELLS IN LYMPH

(75) Inventors: Kristen Adams, Houston, TX (US); Eva Sevick-Muraca, Montgomery, TX (US)

Correspondence Address: CONLEY ROSE, P.C.;David A. Rose, P. O. BOX 3267, HOUSTON, TX, 77253-3267, US

(73) Assignee: BAYLOR COLLEGE OF MEDICINE, Houston, TX (US)

(21) Appl. No.: 11/844,038

(22) Filed: Aug. 23, 2007

Related U.S. Application Data
(60) Provisional application No. 60/823,476, filed on Aug. 24, 2006.

(51) Int. Cl. A61B 5/00

(52) U.S. Cl. 424/9.6

(57) ABSTRACT

Methods and imaging agents for imaging epithelial cancer cells in the lymphatic system are disclosed herein. In an embodiment, an imaging agent for imaging cancer cells in a lymphatic system comprises a fluorescent dye conjugated to one or more antibodies. The antibodies are capable of specific binding to an epithelial cell adhesion molecule (Ep-CAM). In addition, embodiments of the imaging agent may be administered to the lymphatic system where the disclosed imaging agents may bind to an epithelial cell adhesion molecule. The bound imaging agents may be excited with excitation light to image cancer cells in the lymphatic system.

### CROSS-REFERENCE TO RELATED APPLICATIONS

The present application claims the benefit of U.S. Provisional Application Ser. No. 60/823,476 filed Aug. 24, 2006, herein incorporated by reference in its entirety for all purposes.

### STATEMENT REGARDING FEDERALLY SPONSORED RESEARCH OR DEVELOPMENT

This work was supported by National Institutes of Health (R01 CA112679) and America Cancer Society (RSG-06-213-01-LR).

### BACKGROUND

1. Field of the Invention

This invention relates generally to the field of cancer diagnostics. More specifically, the invention relates to a method of imaging and identifying cancer cells in the lymphatic system through near infrared fluorescence labeling.

2. Background of the Invention

The lymph plexus consists of loosely connected epithelium without the structural integrity of smooth muscle cells for efficient collection of fluid and foreign particles. The plexus is located beneath the epidermis and provides the route of entry into the lymph compartment. From the plexus, fluid, cells and foreign particles travel through lymphatic vessels, to the lymph nodes where the particles are taken up by antigen presenting cells for immune presentation and stimulation. The fluid is then returned to the subclavian vein for reentry into the blood stream. Similar to the blood capillary bed, lymph plexus represents the location of most of the fluid transport.

Overwhelming evidence points to the lymph plexus as the first route of cancer dissemination in the body. Recently, metastatic potential of colon, prostate, breast, lung and head and neck cancers was positively correlated with a receptor/ligand system that is specifically required for lymphangiogenesis. This data implicates primary tumor mediated growth of individual lymphatic vessels for cancer cells, representing a “highway on ramp” for cancer cell dissemination throughout the body. Furthermore, the receptor specific to the lymphatic endothelium has been reported in elevated quantities in certain metastatic cancers. In order to accurately stage the occult carcinoma in lymph it is necessary to conduct a surgical resection of the lymphatic system. Surgical resection and lymphatic disruption is directly related to post-operative complications resulting in lymphedema. This complication requires additional treatment including the possible administration of radiation treatment.

Current lymphatic metastasis imaging protocols include ultrasound (US), magnetic resonance (MRI), and computed tomography (CT). Despite recent advances combined with new extra-, and intracellular contrast agents, these techniques represent only non-specific means primarily useful in identifying enlarged lymphatic nodes. Nuclear imaging protocols lymphoscintigraphy and lymphography, techniques of gamma scintigraphy, possess significantly sensitive detection for the identification of occult, micro, and difficult to detect, nodes. These techniques have implications in improved efficacy of treatment through location and removal of the sentinel node, or first draining node from the tumor site. Positron emission tomography, an alternative nuclear imaging protocol, employed as a means to molecularly image lymph nodes with a non-specific glucose-analog. Administered intravenously, this technique results in high-background signals reducing sensitivity in the detection of occult, micro, and difficult to detect, nodes. Furthermore, macrophage uptake in the lymph rather than cancer cell specific uptake impedes accurate diagnosis of cancer.

Although nuclear imaging protocols are currently standard for locating cancer in lymph at the molecular, optical microscopy techniques are being developed. Near infrared fluorescent optical imaging demonstrates favorable signal to noise ratio (SNR) with equivalent or similar target to background ratios (TBR). Additionally, NIR imaging has shown higher sensitivity, and shorter imaging times due to a theoretically increased number of fluorescent photons compared to gamma photons in nuclear imaging.

Consequently, there is a need for a specific, molecularly targeted imaging agent for delivery into the lymphatic compartment for sensitive detection of epithelial cancers, with minimal background.

### BRIEF SUMMARY

Methods and imaging agents for imaging epithelial cancer cells in the lymphatic system are disclosed herein. Embodiments of the methods utilize a novel imaging agent conjugated antibody against epithelial cancer molecules. Administration of the imaging agent localizes to the lymphatic compartment for the identification of epithelial carcinoma metastasis. Further advantages and features of the methods and the imaging agent will be described in more detail below.

Antibodies are highly specific immuno-response molecules that can be raised against the extra-cellular matrix proteins of any lymphatically distributed metastatic epithelial cancers. When conjugated to near infrared fluorescent molecules, such as a heptamethine carbocyanine, the antibodies allow imaging of any cancerous metastasis in the lymphatic system.

In an embodiment, a method for imaging epithelial carcinoma cells in a lymphatic system under a tissue surface comprises delivering an imaging agent to the lymphatic system. The imaging agent comprises one or more antibodies which specifically bind to an epithelial cell adhesion molecule. The method further comprises illuminating the tissue surface with an excitation light to excite the imaging agent. In addition, the method comprises detecting emissions from the imaging agent to image epithelial carcinoma cells within the lymphatic system.

In another embodiment, an imaging agent for imaging cancer cells in a lymphatic system comprises a fluorescent dye conjugated to one or more antibodies. The antibodies are capable of specific binding to an epithelial cell adhesion molecule (Ep-CAM), a molecule that is typically over-expressed in epithelial cancers.

The molecular specificity of antibodies reduces the probability of a false positive, while concurrently increasing signal to noise, and signal to background. The use of antibodies as the means for locating the dispersed cells increases the accuracy of staging the cancer during medical diagnosis. These advantages exceed current protocols because of the decreased likelihood of other non-specific interactions within the patient's lymph. Furthermore, the imaging can be conducted quickly, without prolonged periods of immobilization required in imaging machinery, or further discomfort to the patient.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates imaging agent specificity for epithelial carcinomas in tissue culture;

FIG. 2 illustrates a detection of epithelial carcinoma implanted in a murine model using an embodiment of the disclosed imaging agents;

FIG. 3 illustrates the difference in fluorescent intensity between axillary lymph nodes with and without epithelial carcinoma metastasis; and

FIG. 4 illustrates a schematic of a system that may be used with embodiments of the imaging agent.

### NOTATION AND NOMENCLATURE

Certain terms are used throughout the following description and claim to refer to particular system components. This document does not intend to distinguish between components that differ in name but not function.

In the following discussion and in the claims, the terms “including” and “comprising” are used in an open-ended fashion, and thus should be interpreted to mean “including, but not limited to . . . .”

As used herein “lymphatic system”, “lymph compartment” and “lymph plexus” refers interchangeably to the structures, and network thereof, that comprise the human lymph system including without limitation, lymph ducts, lymph nodes, lymph vessels, collecting vessels and combinations thereof.

### Detailed Description of the Preferred Embodiments

In an embodiment, a method of imaging epithelial cancer cells comprises delivering or administering an imaging agent which binds to human epithelial cell adhesion molecules maybe to the lymphatic system under a tissue surface of a patient. The imaging agent may be allowed to bind to the human epithelial cell adhesion molecules. The tissue surface may then be illuminated with an excitation light to excite the imaging agent. The imaging agent bound to human epithelial cell adhesion molecules emits fluorescent light which may be detected and thus, allows for imaging of cancerous epithelial cells in the lymphatic system.

The delivery of the agent may by any means known to one skilled in the art such as, but not limited to, orally, sublingually, transdermally, rectally, nasally, or by injection. In preferred embodiments, the imaging agent is delivered via intradermal injection.

In embodiments the imaging agent is a fluorescent dye which is bound or coupled to an antibody. Without being limited by theory, antibodies are immuno-response molecules specifically targeted against an antigen in an animal. Antibodies are created by injecting a solution with isolated antigen into the animal. In embodiments the animal is preferably a mammal. Antigens can be a particular infectious agent, protein, molecule, structure, chemical or compound, without limitations, that is not recognized by the animal's immune system. The antigen thereby illicits an immuno-response and the production of antibodies within the immune system of the animal, for distribution in the circulatory system. After an incubation time of about one to about four weeks, blood serum is collected from the animal. The antibodies produced against the antigen are isolated from the blood serum. Preferably, the fluorescent dye is coupled to one or more antibodies which specifically bind to extra-cellular matrix proteins found on cancer cells. In a specific embodiment, the one or more antibodies are specific to Epithelial Cell Adhesion Molecule (Ep-CAM). The Ep-CAM antigen may be human Ep-CAM, mouse Ep-CAM, rat Ep-CAM, sheep Ep-CAM, or rabbit Ep-CAM. The antibodies are preferably monoclonal antibodies. In addition, the antibodies may be derived from any suitable mammal. Examples include without limitation, human antibodies, mouse antibodies, rat antibodies, horse antibodies, sheep antibodies, etc.

As mentioned above, the imaging agents are fluorescent dyes conjugated to the antibody. These fluorescent dyes are chemical compounds that increase contrast between a targeted tissue and the surrounding organs such as, but not limited to dyes, stains, fluorophores, radiation emitters, or other compounds known to one skilled in the art. The fluorescent dyes are typically fluorophores. Fluorophores are compounds in which molecular absorption of a photon of light results in the emission of a photon of light at a lower energy. The fluorescent dye may comprise an acridine, an anthraquinone, an azamethine, a benzimidazol, a cyanine, an indolenine, a napthalimide, an oxazine, an oxonol, a polyene, a polymethin, a porphin, a squaraine, a styryl a thiazol, a xanthin, other compounds known to one skilled in the art, or combinations thereof. Furthermore, the fluorescent dye may have excitation wavelengths ranging from about 400 nm to about 800 nm, preferably from about 650 nm to about 800 nm, more preferably from about 765 nm to about 800 nm. In preferred embodiments, the fluorophores may have excitation wavelengths in the near-infrared range. In a specific embodiment, the fluorescent dye conjugated to the antibody is a heptamethine carbocyanine or IR-800.

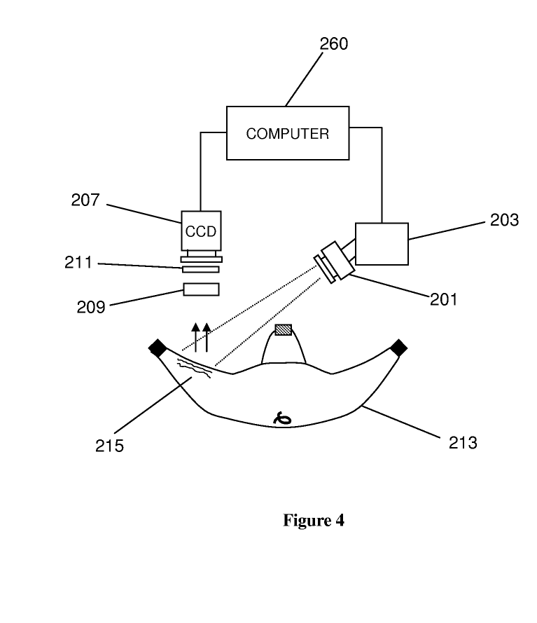

To excite the fluorescent dye in the lymphatic system, an excitation light may be illuminated on the tissue surface over the targeted lymph nodes and/or channels by an excitation light source 201 as shown in FIG. 4. Excitation light source 201 may be any light source known to those of skill in the art. Examples of suitable light sources include without limitation laser diodes, semiconductor laser diodes, gas lasers, light emitting diodes (LEDs), or combinations thereof. In an embodiment, excitation light source may comprise a Gaussian light source. As defined herein, a Gaussian light source is a light source in which the spatial distribution of the emitted light is a Gaussian distribution.

Preferably, the excitation light source 201 is a continuous wave light source which emits a continuous wave light. The light source may emit light having wavelengths ranging from about 700 nm to about 800 nm, preferably from about 725 nm to about 775 nm, more preferably from about 745 nm to about 755 nm. Alternatively, the excitation light source 201 may be a time varying light source. Thus, the intensity of the excitation light source 201 may vary with time. In other words, the excitation light source may emit an intensity-modulated light beam. The intensity modulation of excitation light source may comprise without limitation, sinusoidal, square wave, or ramp wave modulation. In addition, the excitation light source 201 may also be pulsed at certain frequencies and repetition rates. The frequency and repetition rates may also be varied with time. The time variation of the excitation light source may be about 1 to about 3 orders of magnitude of the lifetime of the organic dyes used in conjunction with embodiments of the method.

Upon illumination of the tissue surface by the excitation light, the excitation light penetrates the tissue surface to the lymphatic system and the fluorescent dye administered to the lymphatic system emits fluorescent light. A sensor may be used to detect or sense the emissions from the fluorescent dye. The sensor is preferably capable of detecting fluorescent light emitted from the fluorescent targets and detecting excitation light reflected from the medium. In an embodiment, the sensor may comprise an intensified charge-coupled camera. Other examples of suitable sensor include without limitation, gated or non-gated electron multiplying (EM)-CCD and intensified (I) CCD cameras. The sensor may further comprise any suitable filters or polarizers necessary to measure the appropriate wavelengths of light required for fluorescent optical tomography and imaging.

In further embodiments, it is envisioned that the disclosed methods and imaging agents may be used in conjunction with tomographic imaging to produce three dimensional images of the epithelial cancer cells in the lymphatic system. Tomographic techniques with patterned illumination as disclosed in U.S. patent application Ser. No. 11/688,732, incorporated herein by reference in its entirety for all purposes, may be used to acquire deep tissue images of cancerous epithelial cells.

A radio-emitter, or radiotracer may be also conjugated to the antibody. Radio-emitters are defined herein as an isotope that undergoes radioactive decay yielding gamma or positron emission. In embodiments, the radio-emitter is a gamma-emitter suitable for use in radiography or radioscinitigraphy, more specifically lymphography or lymphoscintigraphy. In embodiments the isotope is from the group of atoms commonly used for medical protocols such as, but not limited to, indium, iodine, cobalt, cesium, cadmium, gallium, germanium, bismuth, manganese, palladium, radium, rubidium, scandium, selenium, tentalium, technetium, thulium, yttrium, ytterbium or others as known to one skilled in the art. In preferred embodiments the gamma-emitter is an isotope of indium.

In another embodiment of the imaging agent, a plurality of fluorescent dyes may be conjugated to one antibody. Multiple fluorescent dyes conjugated to a single antibody are defined herein as multiple labeling. The plurality of fluorescent dyes may be the same or different from one another. Multiple labeling allows multiple imaging protocols to be used as a means of making and verifying a diagnosis. Furthermore, a fluorescent dye may be conjugated to both an antibody and a radiotracer. In one embodiment, the fluorescent dye is a heptamethine carbocyanine, and the radiotracer is an isotope of indium.

FIG. 4 illustrates an example of a system 200 that may be used in conjunction with the disclosed imaging agents and to implement embodiments of the disclosed methods. Briefly, an excitation light source 201 may be mounted on a stepper motor 203 to enable scanning across the tissue surface 213 (i.e. patient) at the desired target tissue region 215. The excitation light may be shaped using a lens 205. Images may be acquired by an intensified CCD camera 207. An image intensifier 209 and filter 211 may be placed in front of the lens of CCD camera 207. Filter 211 may comprise any suitable filter to pass only the emitted light at the excitation wavelength from the organic dye. The captured images may be processed and stored in computer 260. Further examples of and variations on such a system may be found in U.S. Pat. Nos. 5,865,754 and 7,054,002, incorporated herein by reference in their entireties for all purposes.

In addition, the sensor system 200 may modulate, control, attenuate, filter, or otherwise alter the excitation light as known to one skilled in the art. The sensor systems are preferably capable of detecting the imaging agent labeled target in the patient. In an embodiment, the sensor system may comprise a camera, a film, a scintillation counter, a charge coupled device, a multiplier tube, X-ray, MRI or other means of forming an image. The sensor system may further comprise any suitable filters or polarizers necessary to measure the appropriate wavelengths of emission required imaging.

To further demonstrate various illustrative embodiments of the present invention, the following example is provided.

### EXAMPLE

Demonstration of Binding of NIR Dye Labeled, Anti-Human EpCAM to Cancer Epithelial Cells

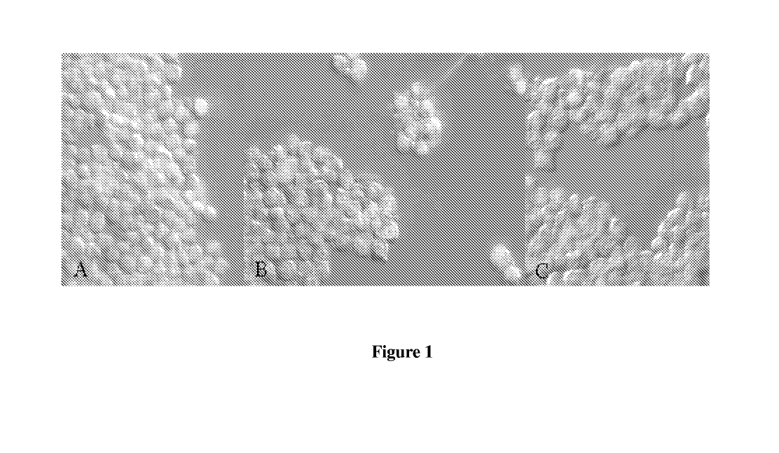

In FIG. 1, the anti-human EpCAM antibody was labeled with NIR fluorescent dye was used to incubate human epithelial cancer cell lines, breast cancers SKBR3 (A) and MDA-MB-231 (B) and human, non-epithelial melanoma cancer cell line, M21 (C). Fluorescence microscopy showed that the imaging conjugate (red color) binds to the epithelial cells and minimally to the non-epithelial cancer cells as determined by the red labeling of the cells in FIG. 1 parts A, and B. The cell nuclei were stained Sytox green for reference.

The results showed that the imaging agent binds to epithelial cells in culture, but not to non-epithelial cell lines. It is important to note that 90% of all human cancers are epithelial and therefore can be targeted with the imaging agent.

Demonstration of NIR-Dye Labeled Anti-EpCAM (Mouse) Targeting to Murine 4T1 Mammary Carcinoma Cells in Axillary Lymph Nodes Associated With Metastatic Spread.

In order to demonstrate the targeting of NIR labeled anti-EpCAM to cancer metastases to the lymph nodes, 4T1 cells were implanted in the left mammary fat pad of the BALB/C and waited for 14 days to allow metastasis to the left axillary node. The 4T1 is an established model of lymph metastasis for small animal study. 50 pmol was administered of anti-EpCAM-NIR in 20 uL intradermally in the forepaws for transit to the axillary lymph node of the mouse. The hypothesis was that the imaging agent would adhere to cancer cells in the left axillary node (to where the 4T1 cells in the left mammary fat pad have metastasized) but will clear more quickly from the right axillary lymph node. While lymphatic clearance is quicker and more efficient in humans, these small animal imaging experiments provided us a method to demonstrate the efficacy of using the EpCAM targeting imaging agent for nodal staging of epithelial cancers.

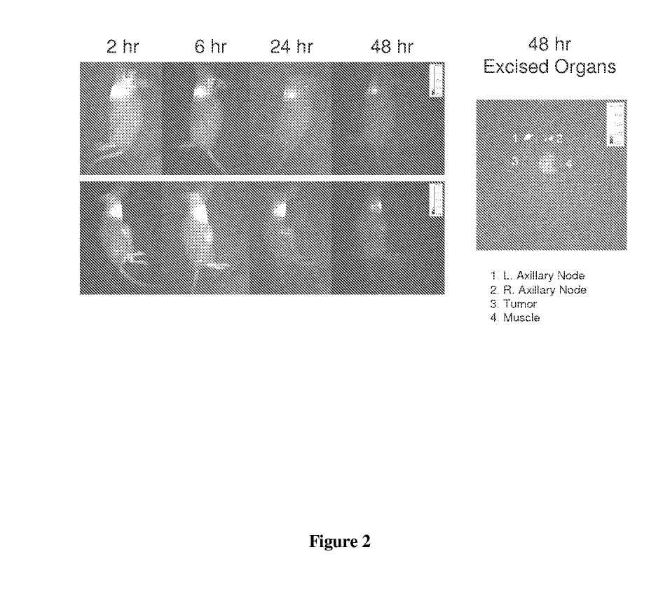

FIG. 2 illustrates the left (top row) and right (bottom row) sides of a single animal injected intradermally with the same amount of imaging agent in the left and right front paws. Upon excision of the organs at 48 hours, the left axillary lymph node was consistently brighter and more fluorescent than the right axillary lymph node as shown in the inset. This was consistent with increased clearance from the cancer-negative lymph node and increased retention in the cancer-positive lymph node. There was considerable variation in the efficient intradermal delivery in mice, but at 24 hours, there was consistently more fluorescence from the cancer positive lymph node (the left axillary lymph node) measured from the animal.

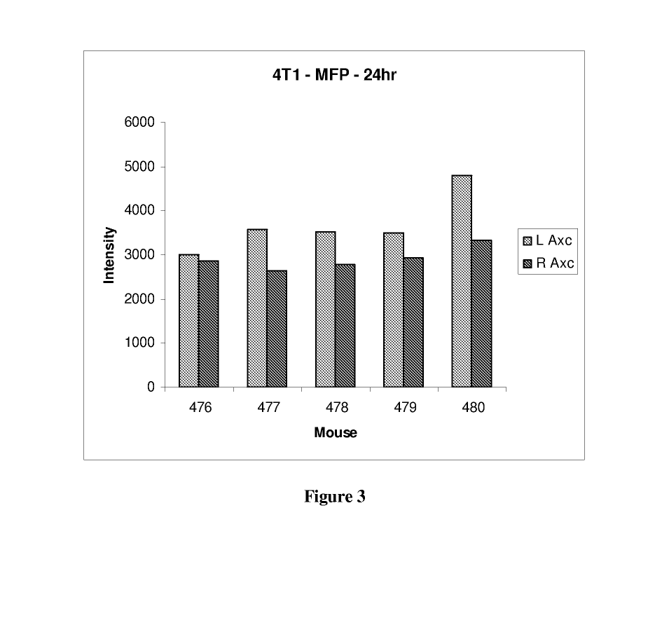

FIG. 3 illustrates the difference in fluorescence intensities of the left and the right axillary lymph node. The histogram shows the in vivo fluorescent intensities from 5 BALBC mice inoculated with 4T1 cancer cells in the mammary fat pad. The cancer positive left axillary lymph node demonstrated markedly higher fluorescence intensities, and was consistent with hypothesis of increased lymphatic clearance of the fluorescent labeled antibodies from the cancer negative, right lymph node.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

The discussion of a reference in the Description of the Related Art is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein.

What is claimed is:

1. A method for imaging epithelial carcinoma cells in a lymphatic system under a tissue surface comprising:
a) delivering an imaging agent to the lymphatic system, wherein the imaging agent comprises one or more antibodies which specifically bind to an epithelial cell adhesion molecule;
b) illuminating the tissue surface with an excitation light to excite the imaging agent;
c) detecting emissions from the imaging agent to image epithelial carcinoma cells within the lymphatic system.

2. The method of claim 1 wherein (a) comprises intradermally injecting the imaging agent.

3. The method of claim 1 wherein the imaging agent comprises a fluorescent dye selected from the group consisting of an acridine, an anthraquinone, an azamethine, a benzimidazol, a cyanine, an indolenine, a napthalimide, an oxazine, an oxonol, a polyene, a polymethin, a porphin, a squaraine, a styryl a thiazol, a xanthin, and combinations thereof.

4. The method of claim 1 wherein the fluorescent dye is heptamethine carbocyanine.

5. The method of claim 3 wherein the fluorescent dye has an excitation wavelength ranging from about 765 nm and about 800 nm.

6. The method of claim 1 wherein the one or more antibodies are coupled to a radiotracer.

7. The method of claim 6 further comprising detecting radiation from the radiotracer to locate epithelial carcinoma cells in the lymphatic system.

8. The method of claim 1 wherein (d) comprises using an intensified charge-coupled camera.

9. The method of claim 1 wherein the one or more lymphatic structures is at least about 1 cm beneath the tissue surface.

10. The method of claim 1 wherein (b) comprises illuminating the tissue surface with an excitation light source selected from group consisting of laser diodes, semiconductor laser diodes, gas lasers, light emitting diodes, and combinations thereof.

11. The method of claim 1 wherein the one or more antibodies are monoclonal antibodies.

12. The method of claim 1 wherein the epithelial cell adhesion molecule (Ep-CAM) comprises human Ep-CAM, mouse Ep-CAM, rat Ep-CAM, sheep Ep-CAM, or rabbit Ep-CAM.

13. The method of claim 1 further comprising using tomographic imaging techniques to provide three-dimensional images of epithelial carcinoma cells in the lymphatic system.

14. An imaging agent for detecting cancer in a lymphatic system comprising:
a fluorescent dye conjugated to one or more antibodies, wherein said antibodies are capable of specific binding to an epithelial cell adhesion molecule (Ep-CAM).

15. The imaging agent of claim 1 further comprising a radiotracer.

16. The imaging agent of claim 14 wherein said radiotracer comprises indium, iodine, cobalt, cesium, cadmium, gallium, germanium, bismuth, manganese, palladium, radium, rubidium, scandium, selenium, tentalium, technetium, thulium, yttrium, ytterbium, or combinations thereof.

17. The imaging agent of claim 1 wherein said one or more antibodies are monoclonal antibodies.

18. The imaging agent of claim 14 wherein the epithelial cell adhesion molecule (Ep-CAM) comprises human Ep-CAM, mouse Ep-CAM, rat Ep-CAM, sheep Ep-CAM, or rabbit Ep-CAM.

19. The imaging agent of claim 14 wherein said one or more antibodies comprises human antibodies, mouse antibodies, rat antibodies, horse antibodies, sheep antibodies, or combinations thereof.

20. The imaging agent of claim 14 comprising a plurality of fluorescent dyes coupled to a single antibody which specifically binds to Ep-CAM, wherein the plurality of fluorescent dyes may be the same or different from each other.
